(19) United States
(12) Patent Application Publication
(10) Pub. No.: US 2024/0315884 A1
(43) Pub. Date: Sep. 26, 2024

(54) PRESSURE-EMBOSSING

(71) Applicant: RKW SE, Mannheim (DE)

(72) Inventor: Leonhard MAIER, Babensham (DE)

(73) Assignee: RKW SE, Mannheim (DE)

(21) Appl. No.: 18/574,823

(22) PCT Filed: Jun. 9, 2022
(86) PCT No.: PCT/EP2022/065639

§ 371 Date: Dec. 28, 2023

(30) Foreign Application Priority Data
Jun. 29, 2021 (DE) 102021116731.0

(51) Int. Cl. A61F 13/514

(52) U.S. Cl. CPC A61F 13/51401; A61F 13/5146; A61F 13/51462; A61F 13/51496; A61F 2013/51415

(57) ABSTRACT

A polyolefinic seepage protection layer in hygiene products with at least one layer having cavities. The layer includes a first area and at least one further area. The first area has a higher volume fraction of cavities than the further area.

### CROSS-REFERENCE TO RELATED APPLICATIONS

This application is a 371 National Phase of PCT/EP2022/065639, filed Jun. 9, 2022, which claims priority from German Patent Application No 10 2021 116 731.0, filed Jun. 29, 2021, both of which are incorporated herein by reference as if fully set forth.

### TECHNICAL FIELD

The invention relates to a polyolefinic seepage protection layer in hygiene products with at least one layer which has cavities.

### BACKGROUND

For the purposes of the invention, the term “hygiene products” refers in particular to diapers for babies and incontinence products for adults, as well as feminine hygiene products such as sanitary towels. Known hygiene products of this type have a protection layer against seepage that extends over the entire product. This seepage protection layer serves to protect the surrounding clothing in order to prevent liquids from leaking out and clothing from becoming contaminated.

The outside of disposable diapers is made of a liquid-impermeable film that prevents excretions from leaking out. The film forms a layer in the form of a flat structure facing away from the backing and is known as a “backsheet”. While gas-impermeable plastic films were used in the early days, breathable films have been used for some time now. These films, which are impermeable to liquids but at the same time permeable to water vapor, significantly improve the wearing comfort of the diaper, as the breathability allows the diaper to dry itself.

In hygiene products for the purposes of the invention, an absorbent body is arranged on a polyolefinic nonwoven film composite. In this respect, the backsheet film carries the absorbent body, which in modern diapers is often enriched with superabsorbent material, and seals it off from the outside. The inside of the hygiene product is usually a topsheet film made of a liquid-permeable material. The topsheet film serves to fix the absorbent core on the inside and increases the wearing comfort of the hygiene product.

Today, a backsheet film should have a textile appearance. This is achieved by a fiber pile on the polyolefin film. Such a combination film is laminated with a non-woven fabric to achieve this textile look.

Another requirement for a polyolefinic seepage protection layer is the minimum tensile strength required for processing the film web on the extremely fast-running diaper production machines (converters). The throughput of the diapers produced is continuously increasing. While speeds of 600 diapers per minute were common a few years ago, today machines for 1000 diapers per minute and more are used. As the line speed increases, higher peak forces act on the seepage protection layer. This places very high demands on the films, especially when folding the finished diapers and the subsequent compression when the air is pressed out of the diaper.

WO 00 2020 225165 A1 describes a modern, breathable film that meets the aforementioned requirements. The specific ball drop impact height of the film is more than 100 mm per gram of polymer per square meter. The film has a water vapor permeability of at least 1000 g/m² in 24 hours. The elongation at break of the film in the machine direction is less than 200%.

In order to create an appealing design, backsheet films are preferably coated with print motifs that identify the manufacturer and are suitable for the target group. The pigments and material combinations used for the printing inks, such as TiO₂, are increasingly in conflict with consumer demands to avoid harmful substances and prevent skin contact with these substances.

### SUMMARY

The object of the invention is to provide a polyolefinic seepage protection layer with a pleasing design. The seepage protection layer should impress with a sophisticated print image and a special type of design and at the same time avoid the use of harmful substances. At the same time, the seepage protection layer should be particularly impermeable to liquids, fulfill the required mechanical properties and ensure a pleasant wearing comfort. Furthermore, the seepage protection layer should be haptically appealing. The seepage protection layer should increase the quality of the hygiene product and meet the requirements for its manufacture in modern processes.

According to the invention, this object is solved by a polyolefinic seepage protection layer in hygiene products. Preferred variants can be found in the claims, the description, the embodiment example and the drawing.

According to the invention, the layer of the seepage protection layer comprises a first area and at least one further area, whereas the first area has a higher volume fraction of cavities than the further area. The seepage protection layer is a sheet-like film with special properties, which has at least one layer. In preferred variants of the invention, the seepage protection layer is formed in two or three layers.

A cavity in the sense of a pore is a very small opening, cavity or depression. The size of these cavities is differentiated and leads to a classification into micro-, meso- and macropores. The presence of pores in at least one layer leads to properties that can be described using the porosity of the layer.

Porosity is a dimensionless measurement and represents the ratio of cavity volume to the total volume of a substance or mixture of substances. It serves as a classifying measure for the actual cavities present.

The first area of the layer has at least several pores that have a dimension in the order of magnitude or larger than the wavelength of visible light. For example, at least some of the pores should have a dimension or diameter of at least 400 nanometers.

The size of the cavities can be determined by measuring the bubble point according to ASTM F-316-80. The cavities may be open-cell cavities or closed-cell cavities. In some embodiments, the pores are closed-cell pores.

The first area of the layer therefore contains micropores, which cause the first area to appear opaque. Opacity is the opposite of transparency. It is a measure of opacity or opacity in percent. In particular, the opacity of a completely opaque layer is 100% and a completely or fully transparent layer has an opacity of 0%.

In particular, opaque areas of polyolefinic seepage protection layers are not transparent in the wavelength range of visible light. Advantageously, the first area of the layer has an opacity according to DIN 53416 of more than 45%, preferably more than 60%, in particular more than 75%.

The further area of the layer, whose average volume of the cavities is smaller than in the first area, has no pores or pore sizes that are smaller than the wavelength of visible light. They cause the further area to appear transparent.

The term translucent refers to either transparent, where the area lets light through and provides a clear view of the layers behind it, or translucent, where the area lets light through but does not provide a clear view of the layers behind it. The translucent area can be colored or colorless to create a pleasing design.

According to the invention, the first and the further area have almost the same number of cavities, although their volume differs significantly. Almost the same number of cavities refers to a difference of less than 10%. In an alternative embodiment of the invention, the number of cavities in the areas differs significantly because the cavities in the wider areas are completely collapsed by the compression molding.

Preferably, the first area has an average volume of cavities higher in the first than in another area. The volume of individual cavities in the areas as well as the total volume of all cavities can differ. In principle, it would be possible for the areas to have a different number of cavities. Under certain circumstances, the areas can have almost the same number of cavities.

In an extremely advantageous embodiment of the invention, the volume fraction of all cavities within the first area is more than 10%, preferably more than 20%, in particular more than 30%. The volume fraction of the first area is larger than that in a further area, wherein the volume fraction of all cavities within the further area is less than 70%, preferably less than 20%, in particular less than 10% of the volume fraction of the first area. In this way, the first area of the layer is opaque, while the further area is translucent to transparent.

To create the cavities, the layer of the seepage protection layer is either β-nucleated, filled with an inorganic filler or both β-nucleated and filled. The nucleating agents and/or the fillers are added to the polymer mixture, preferably blow-extruded to form a film and then stretched to form the cavity structures of the layer according to the invention.

According to the invention, in one variant of the invention, nucleating agents are used in at least one layer of the seepage protection layer. An adjustment of spherulite sizes of the selected polymers with a stretching leads to an opacity which ensures an appealing print design, while at the same time guaranteeing particularly favorable mechanical properties of the seepage protection layer.

With isotactic polypropylene in particular, it is possible to nucleate the α-crystal modification or the β-crystal modification using suitable nucleating agents. α- and β-nucleated polypropylene differ greatly in their mechanical and optical properties. In addition, with a few β-nucleating agents it is also possible to improve the optical properties in such a way that an opaque polypropylene layer can be obtained.

In an advantageous variant of the invention, at least one layer of the seepage protection layer has a proportion of β-spherulite nucleating agent. Here, the proportion of β-spherulite nucleating agent is more than 1 ppm, preferably more than 2 ppm, in particular more than 3 ppm. It has been shown that the use of β-spherulite nucleating agent can be carried out very sparingly and that the proportion is less than 1000 ppm, preferably less than 500 ppm, in particular less than 50 ppm.

A substance known as cinquasia gold, a substituted quinacridone, is preferably suitable as a β-spherulite nucleating agent due to its excellent dispersibility in polymer mixtures. A few ppm of cinquasia gold, preferably 10 ppm of cinquasia gold, added to the polymer mixture in the extruder is sufficient to produce an extremely fine crystalline polypropylene layer. The subsequent stretching of the layer results in the layer having a strong opacity due to the formation of a microporous structure. In principle, all known β-spherulite nucleating agents can be used; quinacridones have proven to be favorable as β-spherulite nucleating agents.

The seepage protection layer is either only stretched in the machine direction (MD) or in both the machine and cross directions (CD). This creates a microporous structure in the layer, which leads to the desired opacity.

The seepage protection layer can, for example, be stretched with a stretch ratio of 1:2 in the machine direction. It is also possible to subject the film web to additional cross stretching (CD).

In a further, advantageous variant of the invention, the layer has an inorganic filler, for example CaCO₃, wherein the proportion of inorganic filler is more than 20% by weight, preferably more than 30% by weight, in particular more than 40% by weight and/or less than 90% by weight, preferably less than 80% by weight, in particular less than 70% by weight.

In an alternative variant of the invention, sufficient opacity is produced without the use of an inorganic filler in the seepage protection layer, preferably without incorporating a white pigment. According to the invention, titanium dioxide in particular can be dispensed with. This is advantageous from both a health and an ecological point of view.

In an extremely advantageous embodiment of the invention, the seepage protection layer has no titanium dioxide and preferably no white pigment to produce opacity. This embodiment meets the requirements of environmental and health protection to a particularly high degree. The seepage protection layer produced in this way enables a hygiene product that is free of harmful substances and allows consumers to feel good when wearing it.

The pronounced opacity of the polypropylene-based seepage protection layer can alternatively also be coated with a print. The seepage protection layer according to the invention is characterized by the fact that it can be produced completely free of inorganic fillers and commonly used white pigments.

Pressure embossing is a process for creating an attractive design without applying a print or print layer to the outside of the seepage protection layer. At temperatures of 120-150° C. and a surface pressure in the range of 5-25 bar, the cavities in the other areas are reduced or even removed. The pressure embossing process leads to the collapse of the pores in the other areas of the layer and can result in the number and volume of the cavities being reduced to zero. In addition, microporous, opaque and transparent pore structures in a tape that has an adhesive and a release coating are known and described in EP 3 007 666 B1.

The surface pressure during pressure embossing is generated by opposing, tempered rollers. The rollers are heated to 70-155° C., preferably to 110-150° C., in particular to 125-145° C., and generate a surface pressure of 5-500 bar, preferably 10-200 bar, in particular 15-50 bar, depending on the film thickness. The pressure embossing of the further area of the layer results in the further area being translucent to transparent, while the first area continues to appear opaque.

During pressure embossing, a first roller is heated and works together with a counter roller, which has the corresponding negatives of the motifs and can also be heated, to produce the embossed motif. The seepage protection layer is subjected to surface pressure in the nip, which causes the cavities in the wider area to collapse. This can be followed by an additional embossing process with further rollers.

The design of the counter roller with corresponding negatives of the print motifs or the design results in a visually appealing design of the seepage protection layer without the application of printing ink and skin contact of possibly damaged fabrics.

The color variation of an appealing design of the seepage protection layer can be designed in a variety of ways. For example, the layer itself can be colored. The collapse of the cavities in the wider area of the layer makes the color nuance of the coloring appear significantly darker.

In addition, the layer behind the first layer can have a coloration, whereby the collapse of the cavities in the wider area of the first layer allows the coloration of the second layer to show through.

In an alternative variant of the invention, a print is applied between a first layer and a second layer of the seepage protection layer, which shows through the transparent areas of the first layer.

In a preferred variant of the invention, the seepage protection layer is formed in three layers, with the middle layer having a coloration that shows through the transparent, wider area of the first layer.

The design created in this way preferably comprises motifs and/or letters and/or numbers and/or characters identifying the manufacturer and/or optical recognition symbols. In addition, patterns and structures can be implemented that are, for example, serpentine, serrated, hexagonal, diamond-shaped, diamond-shaped, parallelogram-shaped, honeycomb-shaped, circular, dot-shaped, star-shaped, canvas-shaped, net-shaped, polygonal, preferably line-shaped, net-shaped, polygonal, preferably triangular, quadrangular, particularly preferably rectangular and square, pentagonal, hexagonal, heptagonal and octagonal, wire-shaped, elliptical, oval and grid-shaped.

In a variant of the invention, the seepage protection layer is irrevocably marked with the aid of heat and/or pressure. This is one way of protecting articles from product piracy. In contrast to printing, which can be removed with a suitable solvent, it is not possible to restore the pore structure destroyed by pressure and heat in such a marked arrangement.

Laser radiation can be used for such an effect to change the pore structure. Alternatively or additionally, the pore structure can be changed by the effect of ultrasound.

In a variant of the invention, the seepage protection layer has an embossed structure.

The embossed structure is preferably based on a recurring and/or regularly arranged pattern. The embossed structure can be a continuous embossed structure, such as a continuous groove structure, or several preferably recurring individual embossed structures.

The seepage protection layer has a water column according to EDANA WSP 80.6 of more than 200 mm, preferably more than 1000 mm, in particular more than 2000 mm, and is therefore extremely impermeable with regard to the passage of liquids.

At the same time, the seepage protection layer is usually designed to be breathable and has a water vapor permeability of at least 500 g/m² in 24 h according to ASTM D6701-01. This makes it possible to create a hygienic article that is particularly comfortable to wear.

In an alternative variant of the invention, the seepage protection layer is not designed to be breathable.

The basis weight of the seepage protection layer is more than 6 g/m², preferably more than 10 g/m², in particular more than 15 g/m² and/or less than 80 g/m², preferably less than 70 g/m², in particular less than 60 g/m².

These basis weights lead to corresponding thicknesses of the seepage protection layer, whereby the thickness is more than 6 μm, preferably more than 10 μm, in particular more than 15 μm and/or less than 85 μm, preferably less than 80 μm, in particular less than 75 μm.

The seepage protection layer according to the invention can be connected to a nonwoven. Furthermore, the layered nonwoven laminate can be provided with elastic ears, so-called front or back ears. These can either be glued to the film or thermobonded.

In a particularly advantageous variant of the seepage protection layer according to the invention, the proportion of polyolefins is more than 40% by weight, preferably more than 55% by weight, in particular more than 70% by weight.

In a preferred variant of the invention, two different polypropylene (PP) components are used to produce the seepage protection layer according to the invention. A lower melting point PP component is combined with a higher melting point polypropylene.

In an extremely advantageous variant of the invention, the layer of the seepage protection layer comprises at least 30% by weight of a lower melting point polypropylene with a DSC melting point between 135 and 145° C. and at least 8% by weight of a higher melting point polypropylene with a DSC melting point between 155 and 165° C.

In a favorable embodiment of the invention, the layer of the seepage protection layer comprises a low-density polyethylene (LDPE) component. The proportion of this LDPE component is preferably more than 0.5% by weight, preferably more than 1% by weight and in particular more than 2% by weight. The LDPE component is preferably present in a proportion of less than 12% by weight, preferably less than 8% by weight, in particular 6% by weight.

To produce the polyolefinic seepage protection layer, a composition according to the invention is coextruded, preferably by means of blow extrusion. Subsequently, in a downstream stretching process, cavities are created at least in the first layer. The areas with different cavity volumes are created by temperature-controlled compression molding of the seepage protection layer in a pair of rollers. The average volume of the cavities is significantly reduced in the areas that are subjected to tempered surface pressure by the rollers, making the areas appear translucent to transparent. The design of the roller pairing arranges the opaque and transparent areas to form a pattern, whereby the patterns are in the form of motifs and/or symbols and/or numbers and/or letters and/or combinations thereof.

The film according to the invention is used as a protection layer against seepage in hygiene products, in particular in disposable diapers, incontinence products and feminine hygiene products.

### BRIEF DESCRIPTION OF THE DRAWINGS

Further advantages and features of the invention are apparent from the description of embodiments with reference to drawings and from the drawings themselves. Shown are:

FIG. 1 a roller arrangement for pressure embossing the seepage protection layer, and

FIG. 2 a roller arrangement for pressure embossing and embossing the seepage protection layer.

### DETAILED DESCRIPTION

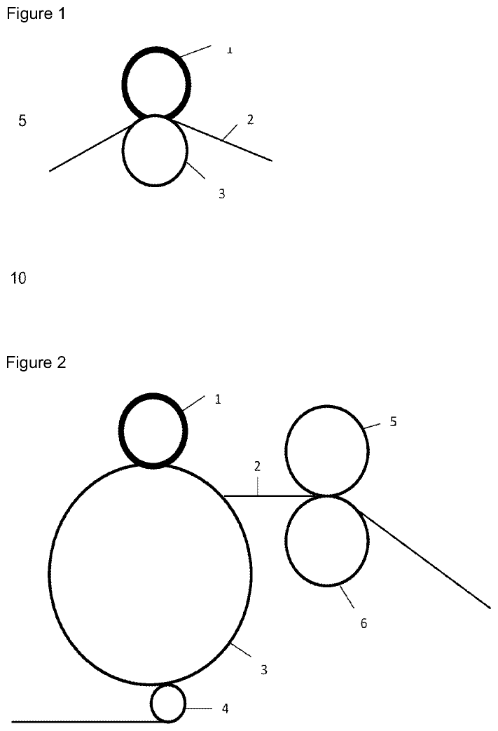

FIG. 1 shows a roller arrangement for pressure embossing the seepage protection layer 2 according to the invention, in which a heating roller 3 and a roller with print motifs 1 act on the seepage protection layer 2 by heating and simultaneous surface pressing in order to cause the volume fraction of the wider area of the seepage protection layer to collapse. The roller with pressure motifs 1 is arranged vertically above the heating roller 3, with the pressure motifs protruding and forming the areas of surface pressure. The roller with print motifs 1 can also be heated.

FIG. 2 shows a roller arrangement for pressure stamping and embossing the seepage protection layer 2. A guide roller 4 directs the seepage protection layer 2 onto the heating roller 3, which heats the seepage protection layer 2. The roller with print motifs 1 is arranged perpendicular to the heating roller 3, whereby the centering axes of the rollers are aligned perpendicular to each other. The protruding pressure motifs of the roller with pressure motifs 1 generate the surface pressure of the seepage protection layer 2 in the roller gap with the heating roller 3, which significantly reduces the volume fraction in the wider area compared to the first area. As a result, the first area remains opaque and the other area is transparent to translucent. The seepage protection layer 2 also undergoes embossing, which is produced in the nip of an embossing roller 5 with a vertically opposed rubber roller 6.

In the following, the invention is explained by means of an example, without limiting the invention to this example.

The following components are used in this example:
48% calcium carbonate, Imerys Filmlink 400
32% PP random copolymer, Braskem DR 155.01
9.6% calcium carbonate, Omyafilm VL-OM PL 753
6.4% PP block copolymer, Inspire 7056.05
4.0% LDPE, DOW 310 E,
1 ppm blue pigment.

The filler used is an inorganic filler in the form of calcium carbonate, preferably with a particle size of 0.8 to 2 μm.

Braskem DR 155.01 is a PP random copolymer. This PP has a melt flow index of 1.7 g/10 min (at 230° C./2.16 kg) according to ASTM D1238 and a DSC melting point of 142° C.

The Inspire 7056.05 is the second PP component. The melt flow index is 3.0 g/10 min (at 230° C., 2.16 kg) in accordance with ISO 1133.

The LDPE DOW 310 E has a density of 0.924 g/cm³. The melt flow index is 0.7 g/10 min (at 190° C., 2.16 kg) in accordance with ISO 1133.

To produce the film according to the invention, the polymer components are heated with the mineral fillers in an extruder, for example a compounding extruder, to a temperature significantly above the melting temperature of the polymer components (for example above 200° C.) and fused together.

This is followed by blow extrusion according to the invention. In the blow extrusion process, a blown tube is formed. The three-layer tubular film formed can be laid flat on top of one another and slit open at the ends to form two film webs.

An inflation ratio of 1:2.5 is used in the blown film process.

The primary basis weight of the film is 40 g/m².

In the subsequent monoaxial stretching process, the film is stretched by a total of 100% in the machine direction.

This results in a film weight of 22 g/m².

The blue-colored seepage protection layer is embossed at 135° C. and with a surface pressure of 20 bar. As a result, the opaque area of the film appears light blue, while the pressure-embossed area appears dark blue and a two-dimensional print motif of the seepage protection layer is formed. In this example, the letters RKW are highlighted in dark blue.

The seepage protection layer has a water vapor permeability according to EDANA WSP 80.6 of 1000 g/m² in 24 hours.

1. A polyolefinic seepage protection layer for hygiene products, the polyolefinic seepage protection layer comprising:
a layer having cavities; and
the layer includes a first area and at least one further area, whereas said first area has a higher volume fraction of cavities than said at least one further area.

2. The seepage protection layer as claimed in claim 1, wherein, the volume fraction of all of the cavities within the first area is more than 10%.

3. The seepage protection layer as claimed in claim 1, wherein the volume fraction of all of the cavities within the further area is less than 70% of the volume fraction of the first area.

4. The seepage protection layer as claimed in claim 1, wherein a water column according to EDANA WSP 80.6 is more than 200 mm.

5. The seepage protection layer as claimed in claim 1, wherein the layer comprises a β-nucleated layer.

6. The seepage protection layer as claimed in claim 1, wherein the layer comprises a filler, a proportion of inorganic filler being more than 20% by weight and less than 90% by weight.

7. The seepage protection layer as claimed in claim 1, wherein the layer does not include any inorganic filler.

8. The seepage protection layer as claimed in claim 1, wherein the layer comprises a stretched layer.

9. The seepage protection layer as claimed in claim 1, wherein the seepage protection layer has a water vapour permeability of more than 500 g/m² in 24 h according to ASTM D6701-01.

10. The seepage protection layer as claimed in claim 1, wherein at least one of the layer or a further layer has a proportion of coloured pigments.

11. The seepage protection layer as claimed in claim 1, wherein a print is arranged behind the layer.

12. The seepage protection layer as claimed in claim 1, wherein a basis weight of the seepage protection layer is more than 6 g/m² and less than 80 g/m².

13. The seepage protection layer as claimed in claim 1, wherein the layer
has at least 30% by weight of a lower melting point polypropylene with a DSC melting point between 135 and 145° C., and
has at least 8% by weight of a higher melting point polypropylene with a DSC melting point between 155 and 165° C.

14. The seepage protection layer as claimed in claim 1, wherein the layer comprises at least 3% by weight of an LDPE.

15. The seepage protection layer as claimed in claim 1 wherein the areas are arranged in a pattern, said patterns comprising at least one of motifs, symbols, numbers, or letters.

16. The seepage protection layer as claimed in claim 1, wherein the areas have a marking by at least one of laser radiation or ultrasound.
